(19) United States
(12) Patent Application Publication
(10) Pub. No.: US 2026/0327280 A1
(43) Pub. Date: Oct. 22, 2026

(54) MUTATED AND WILD TYPE HUMANIN PROTEINS TO IMPROVE CAR-T OR TIL ADOPTIVE CELL THERAPY

(71) Applicant: H. LEE MOFFITT CANCER CENTER AND RESEARCH INSTITUTE, INC., Tampa, FL (US)

(72) Inventors: Reginald "Chip" ATKINS, Tampa, FL (US); Frederick LOCKE, Tampa, FL (US)

(21) Appl. No.: 19/485,559

(22) PCT Filed: May 20, 2024
(86) PCT No.: PCT/US2024/030226

§ 371 Date: Nov. 18, 2025

Related U.S. Application Data
(60) Provisional application No. 63/503,244, filed on May 19, 2023.
(60) Provisional application No. 63/507,512, filed on Jun. 12, 2023.

(51) Int. Cl. C07K 14/47; A61K 40/11; A61K 40/13; A61K 40/15; A61K 40/17; A61K 40/31; A61K 40/41; C07K 16/00; C12N 5/0781; C12N 5/0783; C12N 5/0786

(52) U.S. Cl. CPC C07K 14/47; A61K 40/11; A61K 40/13; A61K 40/15; A61K 40/17; A61K 40/31; A61K 40/41; C07K 16/00; C12N 5/0635; C12N 5/0636; C12N 5/0645; C12N 5/0646; C12N 2510/00

(57) ABSTRACT

Disclosed are engineered isoforms of humanin and the humanin like 8, chimeric antigen receptor cells, engineered tumor infiltrating lymphocytes, and marrow infiltrating lymphocytes comprising said isoforms. Also disclosed are methods of using the engineered isoforms chimeric antigen receptor cells, engineered tumor infiltrating lymphocytes, and marrow infiltrating lymphocytes in methods of treating a neurodegenerative disease.

### II. CROSS REFERENCE TO RELATED APPLICATIONS

This application claims the benefit of U.S. Provisional Application No. 63/503,244, filed on May 19, 2023 and U.S. Provisional Application No. 63/507,512, filed on Jun. 12, 2023, applications which are incorporated herein by reference in their entireties.

### I. STATEMENT REGARDING FEDERALLY SPONSORED RESEARCH OR DEVELOPMENT

This invention was made with Government support under Grant No. CA201594 awarded by the National Institutes of Health. The Government has certain rights in the invention.

### REFERENCE TO SEQUENCE LISTING

The sequence listing submitted on May 20, 2024, as an .XML file entitled “10110-446WO1_ST26.XML” created on May 17, 2024, and having a file size of 12,288 bytes is hereby incorporated by reference pursuant to 37 C.F.R. § 1.52(e)(5).

### III. BACKGROUND

Humanin (HN) is 24-amino acid mitochondria-associated peptide. Since its initial discovery over a decade ago, a role for HN has been reported in many biological processes such as apoptosis, cell survival, substrate metabolism, inflammatory response, and response to stressors such as oxidative stress, ischemia, and starvation. What are needed are analogs of Humanin for the treatment of neurodegenerative diseases and certain types of cancer.

### IV. SUMMARY

Disclosed are methods and compositions related to isoform variants of humanin (MT-RNR2) and humanin like 8 (MT-RNR2L8).

In one aspect, disclosed herein are engineered isoforms of humanin (MT-RNR2) as set forth in SEQ ID NO: 1 comprising a substitution at one or more of residues 9 (such as, for example an L9R substitution as set forth in SEQ ID NO: 2), 10 (such as, for example an L10R substitution as set forth in SEQ ID NO: 3), 11 (such as, for example an L11R substitution as set forth in SEQ ID NO: 4), 19 (such as, for example an P19R substitution as set forth in SEQ ID NO: 5), 20 (such as, for example an V20A substitution as set forth in SEQ ID NO: 6), or 21 (such as, for example an L21R substitution as set forth in SEQ ID NO: 7); wherein when only a single substation at residue 21 is present, the substitution is not an L21A substitution. In one aspect, the engineered isoform can further comprise a L21A substitution and/or a S14G substitution. For example, disclosed herein are engineered isoforms of humanin of any preceding aspect, wherein the isoform comprises a L21A substitution, a S14G substitution, and one or more substitutions selected from the group consisting of L9R, L19R, L11R, P19R, or V20A (such as, for example, as set forth in SEQ ID NO: 8).

Also disclosed herein are engineered isoforms of humanin like 8 (MT-RNR2L8) as set forth in SEQ ID NO: 9 comprising a substitution at one or more of residues 9 (such as, for example an L9R substitution as set forth in SEQ ID NO: 10), 10 (such as, for example an L10R substitution as set forth in SEQ ID NO: 11), 11 (such as, for example an L11R substitution as set forth in SEQ ID NO: 12), 14 (such as, for example a S14G substation), 19 (such as, for example an P19R substitution as set forth in SEQ ID NO: 13), 20 (such as, for example an V20A substitution as set forth in SEQ ID NO: 14), or 21 (such as, for example an L21R substitution as set forth in SEQ ID NO: 15 and/or L21A). For example, disclosed herein are engineered isoforms of humanin like 8, wherein the isoform comprises a L21A substitution, a S14G substitution, and one or more substitutions selected from the group consisting of L9R, L19R, L11R, P19R, or V20A (such as, for example, as set forth in SEQ ID NO: 16).

In one aspect, disclosed herein are chimeric antigen receptor (CAR) immune cells (such as, for example, a T cell (CAR T cell), NK cell (CAR NK cell), NK T cell (CAR NK T cell), B cell (CAR B cell), or macrophage (CARMA cell)) comprising the engineered isoform of humanin or humanin-like 8 of any preceding aspect.

Also disclosed herein are engineered tumor infiltrating lymphocytes (TILs) or engineered marrow infiltrating lymphocytes (MILs) (including, but not limited to tumor infiltrating or marrow infiltrating T cell, NK cell, NK T cell, B cell, or macrophage) comprising the engineered isoform of humanin or humanin-like 8 of any preceding aspect.

In one aspect, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders. Charcot-Marie-Tooth neuropathies, hereditary spastic paraplegias, Friedreich's ataxia, ischemic injury, reperfusion injury, and Tourette's syndrome) as well as stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL of any preceding aspect. For example, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome) as well as stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL comprising engineered isoforms of humanin (MT-RNR2) as set forth in SEQ ID NO: 1 comprising a substitution at one or more of residues 9 (such as, for example an L9R substitution as set forth in SEQ ID NO: 2), 10 (such as, for example an LIOR substitution as set forth in SEQ ID NO: 3), 11 (such as, for example an L11R substitution as set forth in SEQ ID NO: 4), 19 (such as, for example an P19R substitution as set forth in SEQ ID NO: 5), 20 (such as, for example an V20A substitution as set forth in SEQ ID NO: 6), or 21 (such as, for example an L21R substitution as set forth in SEQ ID NO: 7); wherein when only a single substation at residue 21 is present, the substitution is not an L21A substitution. Also, for example, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome) as well as stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL comprising engineered isoforms of humanin like 8 (MT-RNR2L8) as set forth in SEQ ID NO: 9 comprising a substitution at one or more of residues 9 (such as, for example an L9R substitution as set forth in SEQ ID NO: 10), 10 (such as, for example an LIOR substitution as set forth in SEQ ID NO: 11), 11 (such as, for example an L11R substitution as set forth in SEQ ID NO: 12), 14 (such as, for example a S14G substation), 19 (such as, for example an P19R substitution as set forth in SEQ ID NO: 13), 20 (such as, for example an V20A substitution as set forth in SEQ ID NO: 14), or 21 (such as, for example an L21R substitution as set forth in SEQ ID NO: 15 and/or L21A).

### V. BRIEF DESCRIPTION OF THE DRAWINGS

FIG. 1 shows the percentage of PD-1+TIGIT+CD4 (A) and CD8 (B) CAR T with (black) or without (gray) Humanin Like 8 co-expression after manufacture from patient CD3 T cells FACS sorted into CD39 Low or CD39 High populations. Representative FACS plots of populations from A and B.

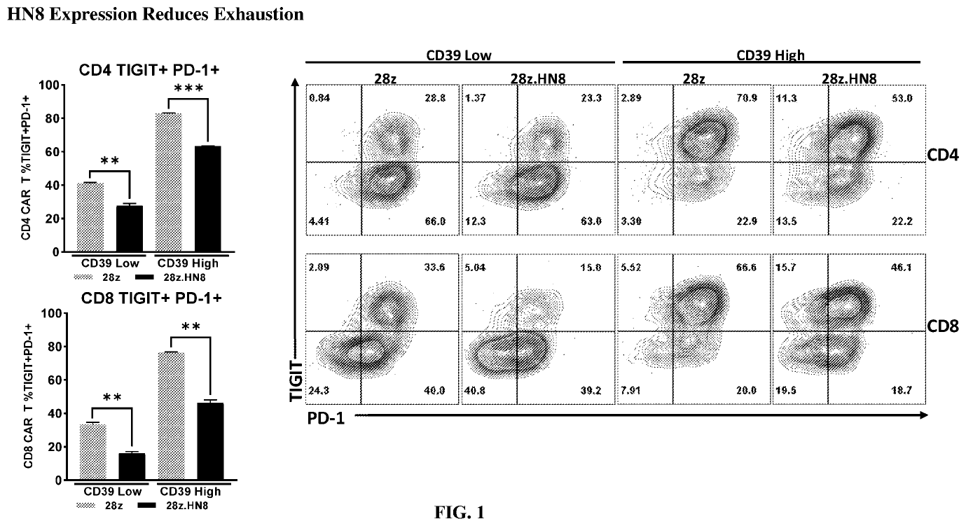

FIG. 2 shows impedance based, real-time cytotoxicity of CAR T with (black) or without (gray) Humanin Like 8 co-expression after manufacture from patient CD3 T cells FACS sorted into CD39 Low or CD39 High populations. CAR T cells were challenged with OCI-Ly3 LBCL target cells (dark gray) at 1:1 and 1:3 CAR T cell to target cell ratios, transduced T cells (light gray).

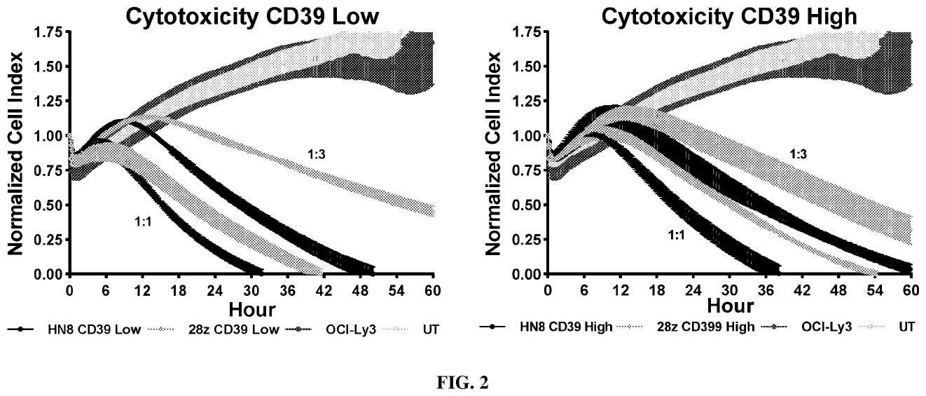

FIG. 3 shows the percentage of apoptotic CAR T cells with (black) or without (gray) Humanin Like 8 co-expression after manufacture from patient CD3 T cells FACS sorted into CD39 Low or CD39 High populations. CAR T cells were stimulated with OCI-Ly3 LBCL target cells at 1:1, 2.5:1, and 5:1, CAR T cell to Target ratios.

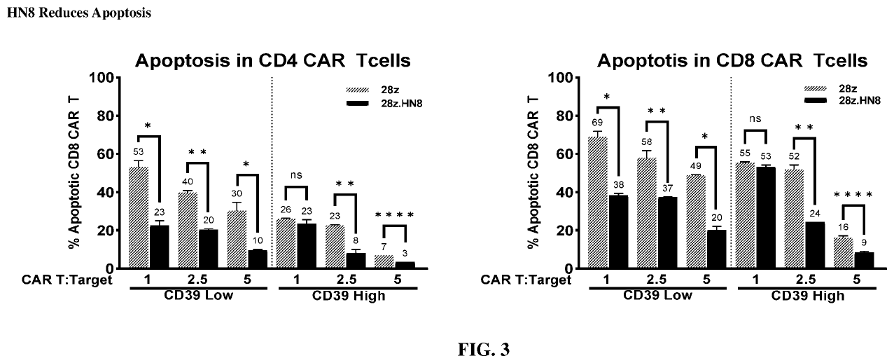

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular MT-RNR2 variant or MT-RNRL8 variant is disclosed and discussed and a number of modifications that can be made to a number of molecules including the MT-RNR2 variant or MT-RNRL8 variant are discussed, specifically contemplated is each and every combination and permutation of MT-RNR2 variant or MT-RNRL8 variant and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Gene 1: MT-RNR2, uniprot Q8IVG9, (a.k.a. Humanin). WT MT-RNR2 comprises the 24 amino acid sequence MAPRGFSCLLLLTSEIDLPVKRRA (SEQ ID NO: 1) where the red residues were targeted for mutation in the following variants. The S14G enhances cytoprotective potency. The K21A/R prevents ubiquitination, increasing protein concentration, and the L9/10/11R, P19R, and V20A substitutions all prevent secretion. Variants include individually and combined mutations mentioned above

MT-RNR2 Variant 1: S14G is shown to enhance cytoprotective effect and has the amino acid sequence: MAPRGFSCLLLLTGEIDLPVKRRA (SEQ ID NO: 2)

MT-RNR2 Variant 2: L21A/R, is the only lysine available for ubiquitination and proteasomal degradation therefore mutation to alanine or arginine prevents this to increase protein concentration and has the amino acid sequence: MAPRGFSCLLLLTSEIDLPVARRA (SEQ ID NO: 3)

MT-RNR2 Variant 3: L9R prevents secretion and has the amino acid sequence: MAPRGFSCRLLLTGEIDLPVARRA (SEQ ID NO: 4)

MT-RNR2 Variant 4: L10R prevents secretion and has the amino acid sequence: MAPRGFSCLRLLTGEIDLPVARRA (SEQ ID NO: 5)

MT-RNR2 Variant 5: L11R prevents secretion and has the amino acid sequence: MAPRGFSCLLRLTGEIDLPVARRA (SEQ ID NO: 6)

MT-RNR2 Variant 6: combined S14G and L21A with secretion has the amino acid sequence: MAPRGFSCLLLLTGEIDLPVARRA (SEQ ID NO: 7)

MT-RNR2 Variant 7: combined S4G, L21A, L9R or any of the single secretory leucine to arginine substitutions at L9/10/11, P19R, or V20A to enhance potency while preventing secretion. The variant shown in SEQ ID NO: 8 (MAPRGFSCRLLLTGEIDLPVARRA) is a representative example and not exhaustive for all possible combinations.

Gene 2: MT-RNR2L8, (a.k.a. Humanin Like 8) differs from Humanin at residue 12, where there is a Serine instead of Leucine. WT MT-RNR2 comprises the 24 amino acid sequence MAPRGFSCLLLSTSEIDLPVKRRA (SEQ ID NO: 9)—red residues are targeted for mutation in the following variants.S14G enhances cytoprotective potency. K21A/R prevents ubiquitination, increasing protein concentration. L9/10/11R, P19R, and V20A substitutions all prevent secretion. Variants include individually and combined mutations mentioned above.

MT-RNR2L8 Variant 1: S14G is shown to enhance cytoprotective effect and has the amino acid sequence: MAPRGFSCLLLSTGEIDLPVKRRA (SEQ ID NO: 10).

MT-RNR2L8 Variant 2: L21A/R, Lysine 21 is the only lysine available for ubiquitination and proteasomal degradation therefore mutation to alanine or arginine prevents this to increase protein concentration and has the amino acid sequence: MAPRGFSCLLLSTSEIDLPVARRA (SEQ ID NO: 11).

MT-RNR2L8 Variant 3: L9R prevents secretion and has the amino acid sequence: MAPRGFSCRLLSTGEIDLPVARRA (SEQ ID NO: 12).

MT-RNR2L8 Variant 4: L10R prevents secretion and has the amino acid sequence: MAPRGFSCLRLSTGEIDLPVARRA (SEQ ID NO: 13).

MT-RNR2L8 Variant 5: L11R prevents secretion and has the amino acid sequence: MAPRGFSCLLLSTGEIDLPVARRA (SEQ ID NO: 14).

MT-RNR2L8 Variant 6: combined S14G and L21A with secretion has the amino acid sequence: MAPRGFSCLLLSTGEIDLPVARRA (SEQ ID NO: 15).

MT-RNR2L8 Variant 7: combined S14G, L21A, L9R or any of the single secretory leucine to arginine substitutions at L9/10/11, P19R, or V20A to enhance potency while preventing secretion. The variant shown in SEQ ID NO: 16 (MAPRGFSCRLLSTGEIDLPVARRA) is a representative example and not exhaustive for all possible combinations.

In one aspect, disclosed herein are engineered isoforms of humanin (MT-RNR2) as set forth in SEQ ID NO: 1 comprising a substitution at one or more of residues 9 (such as, for example an L9R substitution as set forth in SEQ ID NO: 2), 10 (such as, for example an L10R substitution as set forth in SEQ ID NO: 3), 11 (such as, for example an L11R substitution as set forth in SEQ ID NO: 4), 19 (such as, for example an P19R substitution as set forth in SEQ ID NO: 5), 20 (such as, for example an V20A substitution as set forth in SEQ ID NO: 6), or 21 (such as, for example an L21R substitution as set forth in SEQ ID NO: 7); wherein when only a single substation at residue 21 is present, the substitution is not an L21A substitution. In one aspect, the engineered isoform can further comprise a L21A substitution and/or a S14G substitution. For example, disclosed herein are engineered isoforms of humanin of any preceding aspect, wherein the isoform comprises a L21A substitution, a S14G substitution, and one or more substitutions selected from the group consisting of L9R, L19R, L11R, P19R, or V20A (such as, for example, as set forth in SEQ ID NO: 8).

Also disclosed herein are engineered isoforms of humanin like 8 (MT-RNR2L8) as set forth in SEQ ID NO: 9 comprising a substitution at one or more of residues 9 (such as, for example an L9R substitution as set forth in SEQ ID NO: 10), 10 (such as, for example an L10R substitution as set forth in SEQ ID NO: 11), 11 (such as, for example an L11R substitution as set forth in SEQ ID NO: 12), 14 (such as, for example a S14G substation), 19 (such as, for example an P19R substitution as set forth in SEQ ID NO: 13), 20 (such as, for example an V20A substitution as set forth in SEQ ID NO: 14), or 21 (such as, for example an L21R substitution as set forth in SEQ ID NO: 15 and/or L21A). For example, disclosed herein are engineered isoforms of humanin like 8, wherein the isoform comprises a L21A substitution, a S14G substitution, and one or more substitutions selected from the group consisting of L9R, L19R, L11R, P19R, or V20A (such as, for example, as set forth in SEQ ID NO: 16).

In one aspect, disclosed herein are chimeric antigen receptor (CAR) immune cells (such as, for example, a T cell (CAR T cell), NK cell (CAR NK cell), NK T cell (CAR NK T cell), B cell (CAR B cell), or macrophage (CARMA cell)) comprising any of the engineered isoforms of humanin or humanin-like 8 disclosed herein.

Also disclosed herein are engineered tumor infiltrating lymphocytes (TILs) or engineered marrow infiltrating lymphocytes (MILs) (including, but not limited to tumor infiltrating or marrow infiltrating T cell, NK cell, NK T cell, B cell, or macrophage) comprising any of the engineered isoform of humanin or humanin-like 8 disclosed herein.

### 1. Homology/Identity

It is understood that one way to define any known variants and derivatives or those that might arise, of the disclosed genes and proteins herein is through defining the variants and derivatives in terms of homology to specific known sequences. For example, SEQ ID NO: 1 sets forth a particular sequence of an wild type humanin protein (MT-RNR2) and SEQ ID NO: 9 sets forth a particular sequence of a humanin like 8 protein (MT-RNR2L8) and SEQ ID NOs: 2-8 and SEQ ID Nos: 10-16 set forth variants of SEQ ID NO: 1 and SEQ ID NO: 9, respectively. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Peptides

### a) Protein Variants

As discussed herein there are numerous variants of the MT-RNR2 protein and MT-RNR2L8 protein that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of MT-RNR2 protein and SEQ ID Nos: 2-8 set forth RNR2 variants and SEQ ID NO: 9 sets forth a particular sequence of a MT-RNRL8 protein and SEQ ID Nos: 10-16 set forth RNR2L8 variants. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein are also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### C. Methods of Treating Neurodegenerative Diseases

The disclosed compositions can be used to treat any neurodegenerative disease. A representative but non-limiting list of neurodegenerative that the disclosed compositions can be used to treat is the following: Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal Muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome. The disclosed compositions can also be used to treat stroke, diabetes, myocardial ischemia and reperfusion, atherosclerosis, amy-otrophic lateral sclerosis, and certain types of cancers or neurological symptoms associated with said indications.

In one aspect, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome) stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL of any preceding aspect. For example, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome) stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL comprising engineered isoforms of humanin (MT-RNR2) as set forth in SEQ ID NO: 1 comprising a substitution at one or more of residues 9 (such as, for example an L9R substitution as set forth in SEQ ID NO: 2), 10 (such as, for example an L10R substitution as set forth in SEQ ID NO: 3), 11 (such as, for example an L11R substitution as set forth in SEQ ID NO: 4), 19 (such as, for example an P19R substitution as set forth in SEQ ID NO: 5), 20 (such as, for example an V20A substitution as set forth in SEQ ID NO: 6), or 21 (such as, for example an L21R substitution as set forth in SEQ ID NO: 7); wherein when only a single substation at residue 21 is present, the substitution is not an L21A substitution. Also, for example, disclosed herein are methods of treating, inhibiting, reducing, decreasing, ameliorating, and/or preventing a neurodegenerative disease (such as, for example, Alzheimer's disease, Ataxia, Amyotrophic lateral sclerosis (ALS), Bell's palsy, Cerebral Palsy, Chronic Traumatic Encephalopathy (TCE), age related dementia, Lewy body dementia (LBD), epilepsy, encephalitis, Guillain-Barre syndrome, Huntington's disease, meningitis, myasthenia gravis, multiple sclerosis, muscular dystrophy (Duchenne and LGMD), Kennedy's disease, spinocerebellar ataxia, spinal muscular atrophy, hereditary motor neuron disease, early onset muscle disorders, Charcot-Marie-Tooth neuropathies, hereditary spastic paraplegias, Friedreich's ataxia, ischemic injury, reperfusion injury, and Tourette's syndrome) stroke, diabetes, myocardial ischemia and reperfusion, atherosclerosis, amy-otrophic lateral sclerosis, and certain types of cancers in a subject comprising administering to the subject a therapeutically effective amount of the chimeric antigen receptor cell, engineered TIL, engineered MIL comprising engineered isoforms of humanin like 8 (MT-RNR2L8) as set forth in SEQ ID NO: 9 comprising a substitution at one or more of residues 9 (such as, for example an L9R substitution as set forth in SEQ ID NO: 10), 10 (such as, for example an L10R substitution as set forth in SEQ ID NO: 11), 11 (such as, for example an L11R substitution as set forth in SEQ ID NO: 12), 14 (such as, for example a S14G substation), 19 (such as, for example an P19R substitution as set forth in SEQ ID NO: 13), 20 (such as, for example an V20A substitution as set forth in SEQ ID NO: 14), or 21 (such as, for example an L21R substitution as set forth in SEQ ID NO: 15 and/or L21A).

### D. Sequences

SEQ ID NO: 1-Gene 1: MT-RNR2, uniprot Q8IVG9, (a.k.a. Humanin) amino acid
sequence
MAPRGFSCLLLLTSEIDLPVKRRA
SEQ ID NO: 2-MT-RNR2 Variant 1: amino acid sequence
MAPRGFSCLLLLTGEIDLPVKRRA (SEQ ID NO: 2)
SEQ ID NO: 3-MT-RNR2 Variant 2: amino acid sequence
MAPRGFSCLLLLTSEIDLPVARRA
SEQ ID NO: 4-MT-RNR2 Variant 3: amino acid sequence
MAPRGFSCRLLLTGEIDLPVARRA
SEQ ID NO: 5-MT-RNR2 Variant 4 amino acid sequence:
MAPRGFSCLRLLTGEIDLPVARRA
SEQ ID NO: 6-MT-RNR2 Variant 5: amino acid sequence
MAPRGFSCLLRLTGEIDLPVARRA
SEQ ID NO: 7-MT-RNR2 Variant 6: amino acid sequence
MAPRGFSCLLLLTGEIDLPVARRA
SEQ ID NO: 8-MT-RNR2 Variant 7 amino acid sequence
MAPRGFSCRLLLTGEIDLPVARRA
SEQ ID NO: 9-Gene 2: MT-RNR2L8, (a.k.a. Humanin Like 8) amino acid sequence
MAPRGFSCLLLSTSEIDLPVKRRA
SEQ ID NO: 10-MT-RNR2L8 Variant 1 amino acid sequence
MAPRGFSCLLLSTGEIDLPVKRRA
SEQ ID NO: 11-MT-RNR2L8 Variant 2 amino acid sequence
MAPRGFSCLLLSTSEIDLPVARRA
SEQ ID NO: 12-MT-RNR2L8 Variant 3 amino acid sequence
MAPRGFSCRLLSTGEIDLPVARRA
SEQ ID NO: 13-MT-RNR2L8 Variant 4 amino acid sequence
MAPRGFSCLRLSTGEIDLPVARRA
SEQ ID NO: 14-MT-RNR2L8 Variant 5 amino acid sequence
MAPRGFSCLLRSTGEIDLPVARRA
SEQ ID NO: 15-MT-RNR2L8 Variant 6 amino acid sequence
MAPRGFSCLLLSTGEIDLPVARRA
SEQ ID NO: 16-MT-RNR2L8 Variant 7 amino acid sequence
(MAPRGFSCRLLSTGEIDLPVARRA)

1. An engineered isoform of humanin (MT-RNR2) as set forth in SEQ ID NO: 1 comprising a substitution at one or more of residues 9, 10, 11, 19, 20, or 21;
wherein when only a single substation at residue 21 is present, the substitution is not an L21A substitution.

2. The engineered isoform of humanin of claim 1, wherein at least one substitution comprises a L9R substitution.

3. The engineered isoform of humanin of claim 1, wherein at least one substitution comprises a L10R substitution.

4. The engineered isoform of humanin of claim 1, wherein at least one substitution comprises a L11R substitution.

5. The engineered isoform of humanin of claim 1, wherein at least one substitution comprises a P19R substitution.

6. The engineered isoform of humanin of claim 1, wherein at least one substitution comprises a V20A substitution.

7. The engineered isoform of humanin of claim 1, wherein at least one substitution comprises a L21R substitution.

8. The engineered isoform of humanin of claim 1, further comprising an L21A substitution.

9. The engineered isoform of humanin of claim 1, further comprising an S14G substitution.

10. The engineered isoform of humanin of claim 9, wherein the isoform comprises a L21A substitution, a S14G substitution, and one or more substitutions selected from the group consisting of L9R, L19R, L11R, P19R, or V20A.

11. An engineered isoform of humanin (MT-RNR2) as set forth in SEQ ID NO: 9 comprising a substitution at one or more of residues 9, 10, 11, 14, 19, 20, or 21.

12. The engineered isoform of humanin of claim 11, wherein at least one substitution comprises a L9R substitution.

13. The engineered isoform of humanin of claim 11, wherein at least one substitution comprises a L10R substitution.

14. The engineered isoform of humanin of claim 11, wherein at least one substitution comprises an L11R substitution.

15. The engineered isoform of humanin of claim 11, wherein at least one substitution comprises a S14G substitution.

16. The engineered isoform of humanin of claim 11, wherein at least one substitution comprises a P19R substitution.

17. The engineered isoform of humanin of claim 11, wherein at least one substitution comprises a V20A substitution.

18. The engineered isoform of humanin of claim 11, wherein at least one substitution comprises a L21R or L21A substitution.

19. The engineered isoform of humanin of claim 18, wherein the isoform comprises a L21A substitution, a S14G substitution, and one or more substitutions selected from the group consisting of L9R, L19R, L11R, P19R, or V20A.

20. A chimeric antigen receptor (CAR) immune cell comprising the engineered isoform of humanin of claim 1.

21. The chimeric antigen receptor immune cell of claim 20, wherein the immune cell is a T cell (CAR T cell), NK cell (CAR NK cell), NK T cell (CAR NK T cell), B cell (CAR B cell), or macrophage (CARMA cell).

22. An engineered tumor infiltrating lymphocyte (TIL) or engineered marrow infiltrating lymphocyte (MIL) comprising the engineered isoform of humanin of claim 1.

23. The TIL or MIL of claim 22, wherein the lymphocyte is a T cell, NK cell, NK T cell, B cell, or macrophage.

24. A method of treating a neurodegenerative disease in a subject comprising administering to the subject the chimeric antigen receptor cell of claim 20 or the engineered TIL or engineered MIL of claim 22.


SEQUENCE LISTING (16 sequences)

SEQ ID NO: 1 - protein, 24 aa, synthetic construct
MAPRGFSCLLLLTSEIDLPVKRRA

SEQ ID NO: 2 - protein, 24 aa, synthetic construct
MAPRGFSCLLLLTGEIDLPVKRRA

SEQ ID NO: 3 - protein, 24 aa, synthetic construct
MAPRGFSCLLLLTSEIDLPVARRA

SEQ ID NO: 4 - protein, 24 aa, synthetic construct
MAPRGFSCRLLLTGEIDLPVARRA

SEQ ID NO: 5 - protein, 24 aa, synthetic construct
MAPRGFSCLRLLTGEIDLPVARRA

SEQ ID NO: 6 - protein, 24 aa, synthetic construct
MAPRGFSCLLRLTGEIDLPVARRA

SEQ ID NO: 7 - protein, 24 aa, synthetic construct
MAPRGFSCLLLLTGEIDLPVARRA

SEQ ID NO: 8 - protein, 24 aa, synthetic construct
MAPRGFSCRLLLTGEIDLPVARRA

SEQ ID NO: 9 - protein, 24 aa, synthetic construct
MAPRGFSCLLLSTSEIDLPVKRRA

SEQ ID NO: 10 - protein, 24 aa, synthetic construct
MAPRGFSCLLLSTGEIDLPVKRRA

SEQ ID NO: 11 - protein, 24 aa, synthetic construct
MAPRGFSCLLLSTSEIDLPVARRA

SEQ ID NO: 12 - protein, 24 aa, synthetic construct
MAPRGFSCRLLSTGEIDLPVARRA

SEQ ID NO: 13 - protein, 24 aa, synthetic construct
MAPRGFSCLRLSTGEIDLPVARRA

SEQ ID NO: 14 - protein, 24 aa, synthetic construct
MAPRGFSCLLRSTGEIDLPVARRA

SEQ ID NO: 15 - protein, 24 aa, synthetic construct
MAPRGFSCLLLSTGEIDLPVARRA

SEQ ID NO: 16 - protein, 24 aa, synthetic construct
MAPRGFSCRLLSTGEIDLPVARRA